(19) United States
(12) Patent Application Publication
(10) Pub. No.: US 2004/0072886 A1
(43) Pub. Date: Apr. 15, 2004

(54) Novel crystalline forms of (S)-N- (1-Carboxy-2-methyl-prop-1-yl) -N-pentanoyl-N- [2' -(1H-tetrazol-5-yl)- biphenyl-4-yl methyl] amine (Valsartan)

(75) Inventors: Buchi Reddy Reguri, Hyderabad (IN); Sudhakar Sunkari, Hyderabad (IN)

Correspondence Address: Ladas & Parry, 26 West 61st Street, New York, NY, 10023, US

(73) Assignee: DR. REDDY'S LABORATORIES LIMITED

(21) Appl. No.: 10/414,447

(22) Filed: Apr. 15, 2003

(30) Foreign Application Priority Data
Apr. 15, 2002 (IN) 287/MAS/2002
Apr. 26, 2002 (IN) 322/MAS/2002

(51) Int. Cl. A61K 31/41; C07D 257/02

(52) U.S. Cl. 514/381; 548/252

(57) ABSTRACT

The present invention relates to novel crystalline forms of (S)-N-(1-Carboxy-2-methyl-prop-1-yl)-N-pentanoyl-N-[2′-(1H-tetrazol-5-yl)-biphenyl-4-yl methyl amine (Valsartan) and to processes for their preparation.

Valsartan, chemically described as (S)-N-(1-Carboxy-2-methyl-prop-1-yl)-N-pentanoyl-N-[2′-(1H-tetrazol-5-yl)-biphenyl-4-yl methyl amine, is represented by the following structural formula:

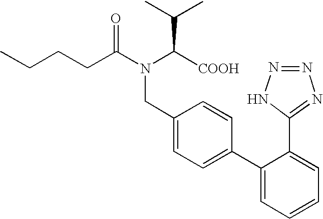

The present invention relates to novel crystalline forms of (S)-N-(1Carboxy-2-methyl-prop-1-yl)-N-pentanoyl-N-[2′-(1H-tetrazol-5-yl)-biphenyl-4-yl methyl amine (Valsartan) and to processes for their preparation.

Valsartan, chemically described as (S)-N-(1-Carboxy-2-methyl-prop-1-yl)-N-pentanoyl-N-[2′-(1H-tetrazol-5-yl)-biphenyl-4-yl methyl amine, is represented by the following structural formula:

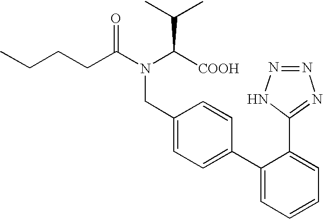

Valsartan, a non-peptide angiotensin—II AT₁ antagonist, inhibits the action of angiotensin—II on its receptors, thus preventing the increase of blood pressure produced by the hormone-receptor interactions. Hence it is used in the treatment of cardiovascular complaints such as hypertension and heart failure.

U.S. Pat. No. 5,399,578 discloses Valsartan, its pharmaceutically acceptable salts and process for their preparation.

WO patent application 02/06253 discloses crystalline, partly crystalline, amorphous and polymorphous forms of specific salts of Valsartan such as monopotassium salt, mono sodium salt, bis-diemethylammonium salt and others.

There is a need for crystalline forms of Valsartan, for preparing pharmaceutical formulations useful for treatment of cardiovascular complaints such as hypertension and heart failure.

The present invention is directed to novel crystalline Forms of Valsartan.

The present invention essentially provides crystalline Form-I and Form-II of Valsartan. The present invention also provides processes for the preparation of novel crystalline Form-I and Form-II of Valsartan by a commercially feasible process very well suited for scale up.

The process for the preparation of novel crystalline Form-I of Valsartan involves dissolution of Valsartan in C₄-C₆ straight or branched chain ketone solvent or a mixture thereof, precipitation from the so formed solution by adding an aliphatic hydrocarbon solvent or mixture thereof, accompanied by isolation and drying to obtain the desired crystalline Form-I of Valsartan.

The process for the preparation of novel crystalline Form-II of Valsartan comprises the dissolution of Valsartan in a ketone solvent and precipitation from the so formed solution by adding an aliphatic hydrocarbon solvent or mixture thereof accompanied by isolation and drying to obtain the desired crystalline polymorph Form-II of Valsartan.

Crystalline Form-I or Form-II of Valsartan of the present invention may exist in unsolvated as well as solvated forms. In general, both unsolvated as well as solvated forms are intended to be encompassed within the scope of the present invention.

### BRIEF DESCRIPTION OF THE ACCOMPANYING DRAWINGS

FIG. 1 is a diagram showing the results of X-ray diffraction of crystalline Form-I of Valsartan.

FIG. 2 is a diagram showing the results of DSC of crystalline Form-I of Valsartan.

FIG. 3 is a diagram showing the results of X-ray diffraction of crystalline Form-II of Valsartan.

FIG. 4 is a diagram showing the results of DSC of crystalline Form-II of Valsartan.

FIG. 5 is a diagram showing the results of X-ray diffraction of the compound obtained by following the reference example.

### DETAILED DESCRIPTION OF THE INVENTION

The present invention provides novel crystalline Form-I and Form-II of Valsartan. The present invention also provides processes for preparation of novel crystalline Form-I and Form-II of Valsartan.

The process for the preparation of crystalline Form-I of Valsartan, comprises;

a) dissolving Valsartan in a C₄-C₆ straight or branched chain ketone solvent at 60-65° C.;

b) adding an aliphatic hydrocarbon solvent accompanied by cooling;

c) isolating and drying the product of step (b) to obtain crystalline Form-I of Valsartan.

In a preferred embodiment hexane is added to the ketone solvent of step a) and then more ketone solvent is added. Preferably the hexane solvent is added at a temperature of 80 to 85° C. Preferably the ratio of hexane to total ketone solvent added in step a) is 2-1:1-2 v/v.

Preferably the ketone solvent is selected from ethyl methyl ketone, methyl isobutyl ketone, methyl isopropyl ketone or diethyl ketone or a mixture thereof. The ratio of Valsartan to straight or branched chain ketone solvents in step a) is 1:1-5 w/v preferably 1:2 w/v.

The aliphatic solvent is a straight or branched chain hydrocarbon or a cyclic hydrocarbon. Preferably the aliphatic hydrocarbon is a C₄-C₈ straight or branched chain hydrocarbon or C₄-C₈ cyclic hydrocarbon. Preferably, the aliphatic solvent is selected from petroleum ether, n-hexane, hexane or cyclohexane or mixture thereof, The ratio of Valsartan to aliphatic hydrocarbon solvent in step b) is 1:1-7 w/v, preferably 1:5 w/v and more preferably 1:3 w/v.

Preferably the solids can be separated by any conventional method, preferably by filtration, decanting or centrifugation; preferably by centrifugation.

Novel crystalline Form-I of Valsartan is characterized by its X-ray diffractogram. The X-ray powder diffraction pattern of crystalline polymorph Form-I of Valsartan was measured on a Bruker Axs, D8 Advance Powder X-ray Diffractometer with Cu K alpha-1 Radiation source. The Crystalline Form-I of Valsartan has X-ray powder diffraction pattern essentially as shown in the Table-1. The X-ray powder diffraction pattern is expressed in the terms of 2θ (in degrees) and percentage intensity (in %).

TABLE 1

| 2-Theta (°) | Intensity (%) |
|---|---|
| 5.415 | 100 |
| 13.145 | 20.1 |
| 17.52 | 16.9 |
| 14.213 | 11.8 |
| 21.09 | 9.0 |
| 14.894 | 8.1 |
| 9.891 | 7.1 |
| 22.1 | 5 |
| 10.726 | 4.4 |

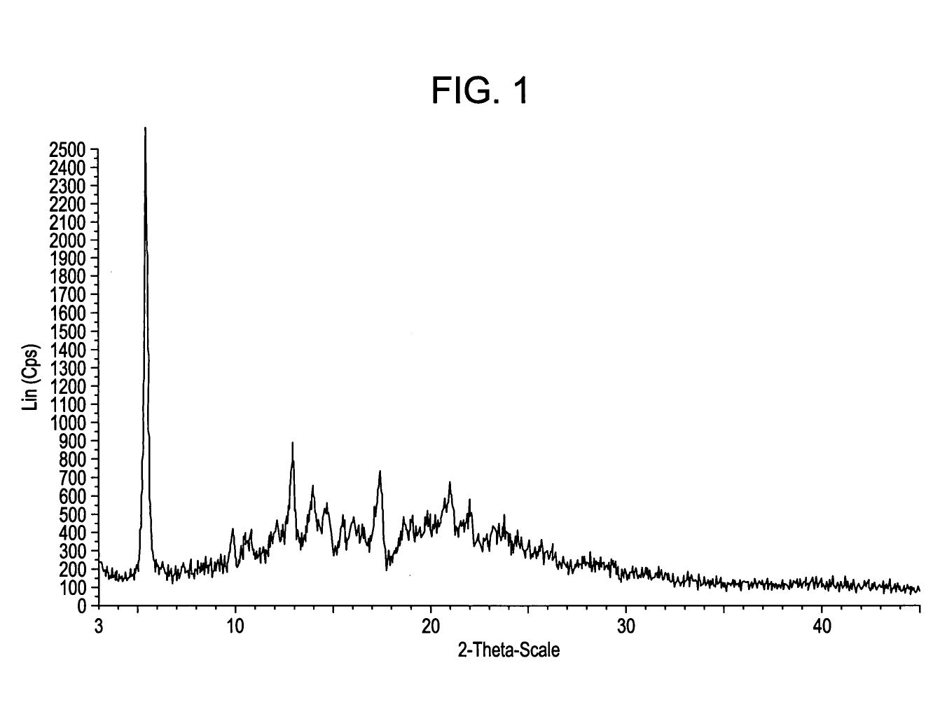

The present invention also provides crystalline Form-I of Valsartan that is characterized by its X-Ray powder diffraction, substantially in accordance with FIG. 1.

Furthermore the present invention provides crystalline Form-I of Valsartan that is characterized by its Differential Scanning Calorimetry thermogram. The Differential Scanning Calorimetry thermogram exhibits a significant endo peak at about 90.24° C.

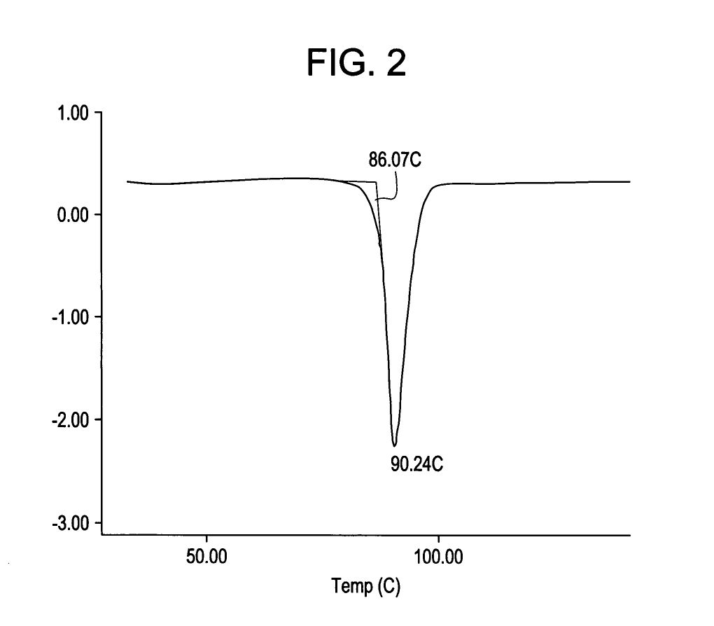

The present invention also provides crystalline Form-I of Valsartan that is characterized by its Differential Scanning Calorimetry thermogram substantially in accordance with FIG. 2.

The present invention further provides crystalline form-I of Valsartan having a visual melting point (capillary tube) in the range of about 80-91° C. The said crystalline form-I of Valsartan is stable white to off-white crystalline powder.

Another aspect of the present invention is to provide novel crystalline Form-II of Valsartan.

The process for the preparation of crystalline Form-II of Valsartan comprises:

i) dissolving Valsartan in a C₄-C₆ ketone solvent at 50-55° C. temperature;

ii) adding an aliphatic hydrocarbon solvent accompanied by cooling;

iii) isolating and drying the product of step (ii) to obtain crystalline Form-II of Valsartan.

In a preferred embodiment hexane is added to the ketone solvent of step i) and then more ketone solvent is added. Preferably the hexane solvent is added at a temperature of 50-55° C. Preferably, the ratio of hexane to total ketone solvent added in step i) is 2-1:1-2 v/v.

The ketone solvent employed in step i) comprises solvents such as methyl propyl ketone.

The ratio of Valsartan to ketone solvent in step i) is 1:1-5 w/v preferably 1:2 w/v. The aliphatic solvent is a straight or branched chain hydrocarbon or a cyclic hydrocarbon. Preferably the aliphatic hydrocarbon is a C₄-C₈ straight or branched chain hydrocarbon or C₄-C₈ cyclic hydrocarbon. Preferably, the aliphatic hydrocarbon is selected from petroleum ether, n-hexane, hexane or cyclohexane or mixture thereof The ratio of Valsartan to aliphatic hydrocarbon solvent in step ii) is 1:1-7 w/v, preferably 1:5 w/v and more preferably 1:3 w/v.

Preferably the solids can be separated by any conventional method, preferably by filtration, decanting or centrifugation; preferably by centrifugation.

Novel crystalline Form-II of Valsartan is characterized by its X-ray diffractogram. The X-ray powder diffraction pattern of crystalline polymorph Form-II of Valsartan was measured on a Bruker Axs, D8 Advance Powder X-ray Diffractometer with Cu K alpha-1 Radiation source.

The Crystalline Form-II of Valsartan has X-ray powder diffraction pattern essentially as shown in the Table-2.

The X-ray powder diffraction pattern is expressed in the terms of 2θ (in degrees) and percentage intensity (in %)

TABLE 2

| 2-Theta (°) | Intensity (%) |
|---|---|
| 5.48 | 100 |
| 6.113 | 82.5 |
| 17.598 | 22.9 |

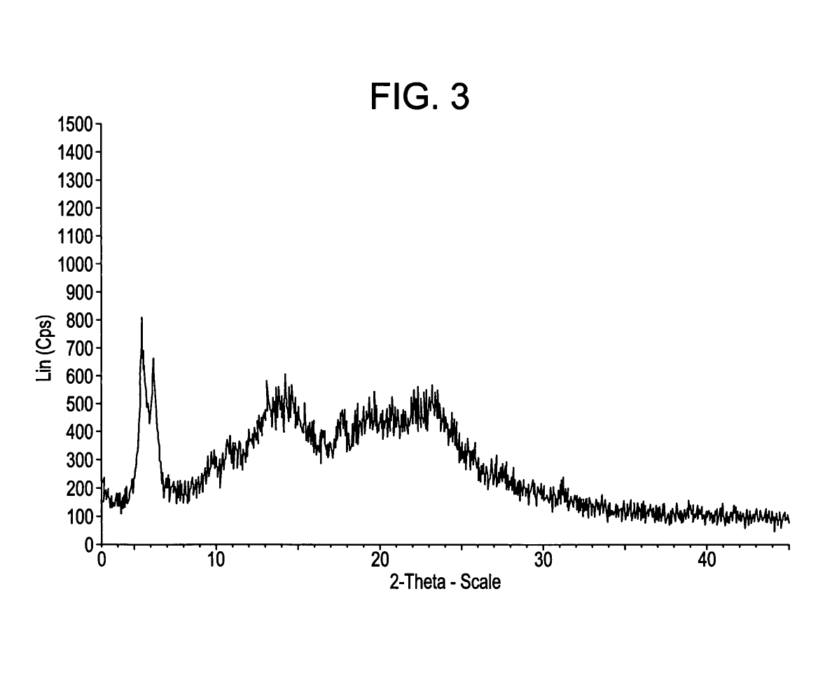

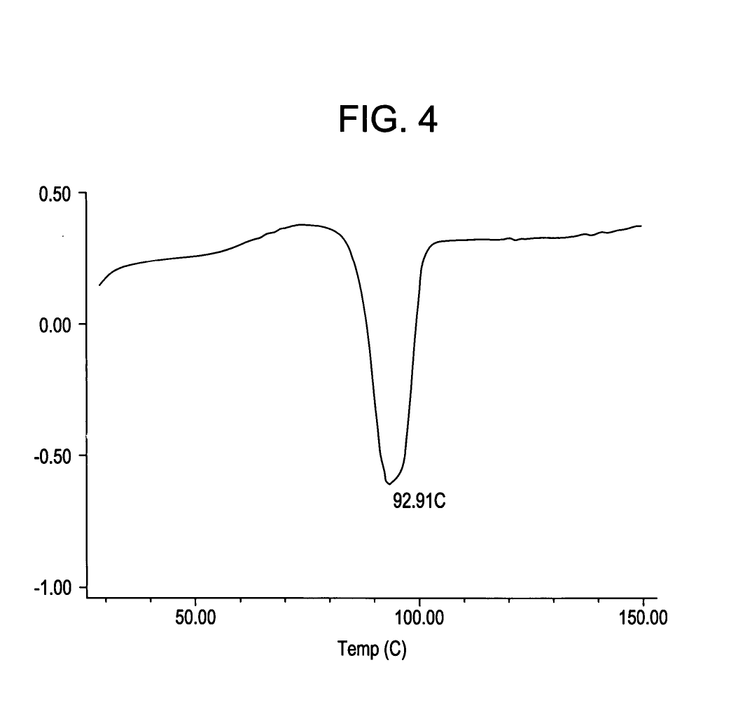

The present invention also provides crystalline Form-II of Valsartan that is characterized by its X-Ray powder diffraction, substantially in accordance with FIG. 3. Furthermore the present invention provides crystalline Form-II of Valsartan that is characterized by its Differential Scanning Calorimetry thermogram. The Differential Scanning Calorimetry thermogram exhibits a significant endo peak at about 92.91° C. The present invention also provides crystalline Form-II of Valsartan that is characterized by its Differential Scanning Calorimetry thermogram substantially in accordance with FIG. 4.

The present invention further provides crystalline Form-II of Valsartan having a visual melting point (capillary tube) in the range of about 91-102° C. The said crystalline Form-II of Valsartan is a stable white to off-white crystalline powder. The Valsartan employed for the preparation of the novel crystalline Form I and Form-II may be obtained by processes disclosed in the prior art.

The invention likewise relates to the use of novel crystalline Form-I and Form-II of Valsartan as angiotensin II antagonist, active substance. In this connection, they can be used, preferably in the form of pharmaceutically acceptable preparations, in a method for the prophylactic and/or therapeutic treatment of the animal or human body, in particular as angiotensin II antagonists.

The invention likewise relates to pharmaceutical preparations which contain novel crystalline Form-I and Form-II of Valsartan as active ingredient and to processes for their preparation.

The pharmaceutical preparations according to the invention which contain the compound according to the invention are those for enteral, such as oral, furthermore rectal, and parenteral administration to (a) warm-blooded animal(s), the pharmacological active ingredient being present on its own or together with a pharmaceutically acceptable carrier. The daily dose of the active ingredient depends on the age and the individual condition and also on the manner of administration.

The novel pharmaceutical preparations contain, for example, from about 10% to about 80%, preferably from about 20% to about 60%, of the active ingredient. Pharmaceutical preparations according to the invention for enteral or parenteral administration are, for example, those in unit dose forms, such as sugar-coated tablets, tablets, capsules or suppositories, and furthermore ampoules. These are prepared in a manner known per se, for example by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores. Suitable carriers are, in particular, fillers, such as sugars, for example lactose, sucrose, mannitol or sorbitol, cellulose preparations and/or calcium phosphates, for example tricalcium phosphate or calcium hydrogen phosphate, furthermore binders, such as starch paste, using, for example, corn, wheat, rice or potato starch, gelatin, tragacanth, methylcellulose and/or polyvinylpyrrolidone, if desired, disintegrants, such as the above mentioned starches, furthermore carboxymethyl starch, crosslinked polyvinylpyrrolidone, agar, alginic acid or a salt thereof, such as sodium alginate; auxiliaries are primarily glidants, flow-regulators and lubricants, for example silicic acid, talc, stearic acid or salts thereof, such as magnesium or calcium stearate, and/or polyethylene glycol. Sugar-coated tablet cores are provided with suitable coatings which, if desired, are resistant to gastric juice, using, inter alia, concentrated sugar solutions which, if desired, contain gum arabic, talc, polyvinylpyrrolidone, polyethylene glycol and/or titanium dioxide, coating solutions in suitable organic solvents or solvent mixtures or, for the preparation of gastric juice-resistant coatings, solutions of suitable cellulose preparations, such as acetylcellulose phthalate or hydroxypropylmethylcellulose phthalate. Colorants or pigments, for example to identify or to indicate different doses of active ingredient, may be added to the tablets or sugar-coated tablet coatings.

Other orally utilizable pharmaceutical preparations are hard gelatin capsules, and also soft closed capsules made of gelatin and a plasticizer, such as glycerol or sorbitol. The hard gelatin capsules may contain the active ingredient in the form of granules, for example in a mixture with fillers, such as lactose, binders, such as starches, and/or lubricants, such as talc or magnesium stearate, and, if desired, stabilizers. In soft capsules, the active ingredient is preferably dissolved or suspended in suitable liquids, such as fatty oils, paraffin oil or liquid polyethylene glycols, it also being possible to add stabilizers.

Suitable rectally utilizable pharmaceutical preparations are, for example, suppositories, which consist of a combination of the active ingredient with a suppository base. Suitable suppository bases are, for example, natural or synthetic triglycerides, paraffin hydrocarbons, polyethylene glycols or higher alkanols. Furthermore, gelatin rectal capsules which contain a combination of the active ingredient with a base substance may also be used. Suitable base substances are, for example, liquid triglycerides, polyethylene glycols or paraffin hydrocarbons.

Suitable preparations for parenteral administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilizers.

The dose of the active ingredient depends on the warm-blooded animal species, the age and the individual condition and on the manner of administration. In the normal case, an approximate daily dose of about 10 mg to about 350 mg is to be estimated in the case of oral administration for a patient weighing approximately 75 kg. For other types of administration, the preferred daily dose is between 0.1 mg to 1000 mg per kilogram.

The following examples illustrate the invention described above; however, they are not intended to limit its extent in any manner.

### Reference Example

Preparation of (S)-N-(1-carboxy-2-methyl-prop-1-yl)-N-pentanoyl-N-[2′-(1H-tetrazol-5-yl)-biphenyl-4-yl methyl] amine (Valsartan)

N-Valeryl-N-[(2′-cyanobiphenyl-4-yl) methyl]-(L)-valine methyl ester (51.5 kg), tributyl tin chloride (61.9 kg), sodium azide (16.5 kg) were added to xylene (258 lit) and stirred for 1-2 hours at a temperature of 25-35° C. then heated the mass to reflux and stirred till the reaction substantially completes. Cool the mass to 25-35° C. and 10% sodium hydroxide solution (250 lit.) was added and further stirred for 24-30 hours.

The aqueous layer was separated from the resulting biphasic solution and washed with toluene (52×2 lit.). The pH of the aqueous layer was adjusted towards neutral with acetic acid (115 lit.) and washed with chloroform (52×2 lit.). The pH of the aqueous layer was further lowered with acetic acid (20 lit.) and extracted the compound into dichloromethane (220×1+110×1). The combined organic layer was successively washed with water, 5% sodium chloride solution and dried over anhydrous sodium sulphate. The solvent from the reaction solution was completely distilled off and triturated the resulting oily mass with hexane to yield the crude Valsartan, which was recrystallised in dichloromethane followed by ethyl acetate to afford sufficient pure Valsartan, which is having an amorphous pattern by its X-ray diffractogram (Yield: 8.8 kgs).

### Preparation of Crystalline Form-I of Valsartan

Valsartan (25.0 g) was dissolved in methyl isobutyl ketone (50.0 ml) at a temperature of 60-65° C. Further hexane (60 ml) was slowly added at a temperature of 80 to 85° C. The mixture was further heated to a temperature of 80-85° C. followed by addition of Methyl isobutyl ketone (10.0 ml). The reaction mixture was then cooled to a temperature of 25-35° C. and left overnight to crystallize to obtain a solid mass. The isolated crystalline solid mass was filtered, washed with hexane (10.0 ml) and dried at 50-70° C. to a constant weight to obtain 23.0 g of the desired crystalline Form-I of Valsartan. m.p.: 80.3-87.6° C.

### Preparation of Crystalline Polymorph Form-II of Valsartan

Valsartan (25.0 grams) was dissolved in methyl propyl ketone (50.0 ml) at a temperature of 50° C. further Hexane (55.0 ml) was slowly added at a temperature of 50-55° C. followed by Methyl propyl Ketone (2.0 ml) and cooled the mass to a temperature of 25-35° C. and kept aside for 30-60 minutes to crystallize the solid mass. The isolated crystalline solid mass was filtered, washed with hexane (15.0 ml) and dried at 60-65° C. to a constant weight to obtain 23.0 gm of the desired crystalline Form-II of Valsartan. m.p.: 91.5-95.5° C.

### DETAILED DESCRIPTION OF THE ACCOMPANYING DRAWING

FIG. 1 is characteristic X-ray powder diffraction pattern of Crystalline Form-I Valsartan. Vertical axis: Intensity (CPS); Horizontal axis: 2θ (degrees). The sample was scanned between 0 to 450. The significant 2θ values (in degrees) obtained are at about 5.415, 13.145, 17.52, 14.213, 21.09, 14.894, 9.891, 22.1, 10.726

FIG. 2 is Differential Scanning Calorimetry thermogram of crystalline Form-I of Valsartan. The heating rate was 5° C./minute. The Differential Scanning Calorimetry thermogram exhibits a single endo peak at about 90.24° C.

FIG. 3 is characteristic X-ray powder diffraction pattern of the novel crystalline Form-II of Valsartan. The sample was scanned between 0 to 45°. Vertical axis: Intensity (CPS); Horizontal axis: 2θ (degrees). The significant 2θ values (in degrees) obtained are 5.48, 6.113 and 17.598 degrees.

FIG. 4 is Differential Scanning Calorimetric Thermogram of novel crystalline Form-II of Valsartan. The heating rate was 5° C./minute. The Differential Scanning Calorimetric Thermogram exhibits a significant endo peak at 92.91° C.

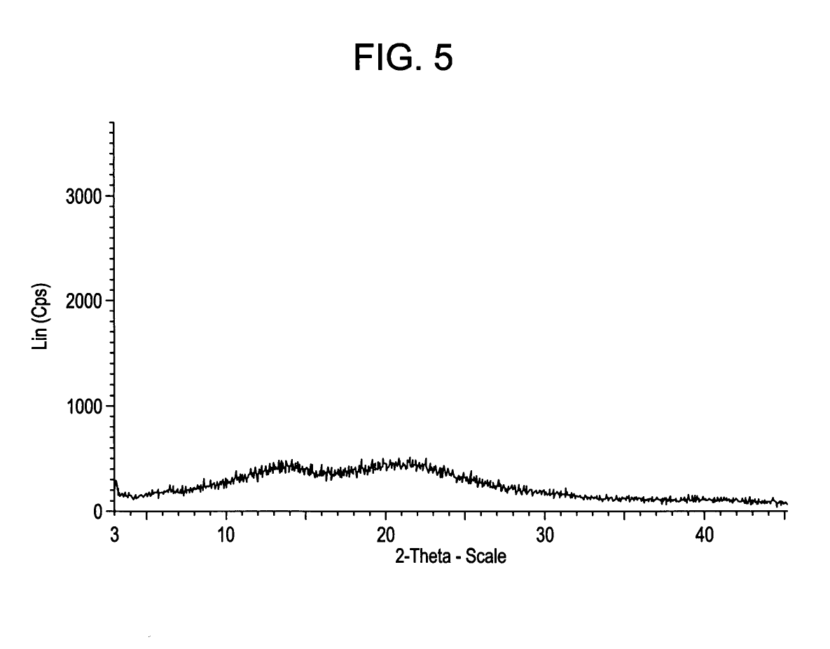

FIG. 5 is characteristic X-ray powder diffraction pattern of Valsartan prepared as per reference example. It shows a plain halo with no peaks, which is a characteristic nature of amorphous form.

1. A novel crystalline Form-I of (S)-N-(1-carboxy-2-methyl-prop-1-yl)-N-pentanoyl-N-[2′-(1H-tetrazol-5-yl)-biphenyl-4-yl methyl] amine.

2. The crystalline Form-I according to claim 1 characterized by an X-ray powder diffraction pattern with peaks at about 2θ values of 5.415, 9.891, 10.726, 13.145, 14.213, 14.894, 17.52, 21.09 and 22.1 degrees.

3. The crystalline Form-I according to claim 1, characterized by XRD pattern substantially in accordance with FIG. 1

4. The crystalline Form-I according to claim 1, having a differential scanning calorimetry thermogram, which exhibits a characteristic endo peak at about 90.24° C.

5. The crystalline Form-I according to claim 4, characterized by DSC pattern substantially in accordance with FIG. 2.

6. The crystalline Form-I according to claim 1, having a melting point in the range of about 80-91° C.

7. A novel crystalline Form-II of (S)-N-(1-carboxy-2-methyl-prop-1-yl)-N-pentanoyl-N-[2′-(1H-tetrazol-5-yl)-biphenyl-4-yl methyl] amine.

8. The crystalline Form-II of Valsartan according to claim 7, characterized by an X-ray powder diffraction pattern with peaks at about 2θ values of 5.48, 6.113 and 17.598 degrees

9. The crystalline Form-II according to claim 7, characterized by XRD pattern substantially in accordance with FIG. 3.

10. The crystalline Form-II according to claim 7, having a differential scanning calorimetry thermogram, which exhibits a characteristic endo peak at about 92.91° C.

11. The crystalline Form-II according to claim 8, characterized by DSC pattern substantially in accordance with FIG. 2.

12. The crystalline Form-II according to any one of claims 7 to 11, having a melting point in the range of about at about 91-102° C.

13. A process for preparation of crystalline polymorph Form-I of (S)-N-(1Carboxy-2-methyl-prop-1-yl)-N-pentanoyl-N-[2′-(1H-tetrazol-5-yl)-biphenyl-4-yl methyl] amine (Valsartan), which comprises:
a) dissolving Valsartan in C₄-C₆ straight or branched chain ketone solvent or a mixture thereof;
b) adding an aliphatic hydrocarbon solvent to the solution of step (a), accompanied by cooling; and
c) isolating and drying the product of step (b) to obtain crystalline Form-I of Valsartan.

14. The process according to claim 13, where in the ratio of Valsartan to C₄-C₆ straight or branched chain ketone solvents or a mixture thereof is 1:1-5 w/v

15. The process according to claim 14, where in the ratio of Valsartan to C₄-C₆ straight or branched chain ketone solvent or a mixture thereof is 1:2 w/v

16. The process according to claim 13, wherein the ratio of Valsartan to aliphatic hydrocarbon solvent is 1:1-7 w/v.

17. The process according to claim 16, wherein the ratio of Valsartan to aliphatic hydrocarbon solvent is 1:3 ratio w/v.

18. The process according to any one of claims 13-15, wherein the ketone solvent is selected from ethyl methyl ketone, methyl isobutyl ketone, methyl isopropyl ketone or diethyl ketone or mixtures thereof.

19. The process according to claim 18, wherein the ketone solvent is methyl isobutyl ketone.

20. The process according to any one of claims 13, 16-17, wherein the aliphatic hydrocarbon solvent is selected from petroleum ether, n-hexane, hexane or cyclohexane or mixtures thereof.

21. The process according to claim 20, wherein the aliphatic hydrocarbon solvent is hexane.

22. A process for preparation of crystalline polymorph Form-II of (S)-N-(1-Carboxy-2-methyl-prop-1-yl)-N-pentanoyl-N-[2′-(1H-tetrazol-5-yl)-biphenyl-4-yl methyl] amine, which comprises:
i) dissolving crude Valsartan in ketone solvent;
ii) adding the aliphatic hydrocarbon solvent to the solution of step (i), accompanied by cooling; and
iii) isolating and drying the product of step (ii) to obtain crystalline Form-II of Valsartan.

23. The process according to claim 22, where in the ratio of Valsartan to ketone solvent is 1:1-5 w/v.

24. The process according to claim 23, where in the ratio of Valsartan to ketone solvent is 1:2 w/v

25. The process according to claim 22, wherein the ratio of Valsartan to aliphatic hydrocarbon solvent is 1:1-7 w/v.

26. The process according to claim 25, wherein the ratio of Valsartan to aliphatic hydrocarbon solvent is 1:1-3 w/v.

27. The process according to any one of claims 22-24, where in the ketone solvent is methyl propyl ketone.

28. The process according to any one of the claims 22, 25-26 wherein the aliphatic hydrocarbon solvent is selected from petroleum ether, n-hexane, hexane or cyclohexane or mixtures thereof.

29. The process according to claim 28, wherein the aliphatic hydrocarbon solvent is hexane.

30. A composition comprising novel crystalline Form of (S)-N-(1-carboxy-2-methyl-prop-1-yl)-N-pentanoyl-N-[2′-(1H-tetrazol-5-yl)-biphenyl-4-yl methyl] amine according to any one of claims 1 to 12 and pharmaceutically acceptable carrier, diluent, excipient, additive, filler, lubricant, binder, stabilizer, solvent or solvate.

31. The composition according to claim 30, in the form of a tablet, capsule, lozenge, powder, syrup, solution, suspension, ointment, or dragee.

32. The composition according to any one of claim 30 or 31, for the treatment of hypertension and heart failure.

33. A method for treating hypertension or heart failure comprising administering an effective amount of crystalline Form of (S)-N-(1-carboxy-2-methyl-prop-1-yl)-N-pentanoyl-N-[2′-(1H-tetrazol-5-yl)-biphenyl-4-yl-methyl] amine according to any one of claims 1-12 and a pharmaceutically acceptable carrier, diluent, excipient, additive, filler, lubricant, binder, stabilizer, solvent or solvate to a patient in need thereof.

34. A medicine for the treatment of hypertension or heart failure comprising an effective amount of crystalline Form of (S)-N-(1-carboxy-2-methyl-prop-1-yl)-N-pentanoyl-N-[2′-(1H-tetrazol-5-yl)-biphenyl-4-yl methyl] amine according to any one of claims 1-12.

35. Use of crystalline Form of (S)-N-(1-carboxy-2-methyl-prop-1-yl)-N-pentanoyl-N-[2′-(1H-tetrazol-5-yl)-biphenyl-4-yl methyl] amine according to any one of claims 1-12 or 30-32 for the preparation of a medicament for the treatment of hypertension or heart failure.
